(19) United States
(12) Patent Application Publication
(10) Pub. No.: US 2024/0033257 A1
(43) Pub. Date: Feb. 1, 2024

(54) USE OF PPAR-DELTA INHIBITOR IN COMBINATION WITH IMMUNOTHERAPEUTIC DRUG FOR PREPARING ANTI-TUMOR DRUG

(71) Applicant: THE FIRST HOSPITAL OF JILIN UNIVERSITY, Changchun (CN)

(72) Inventors: Wentao LIU, Changchun (CN); Yongguang YANG, Changchun (CN); Chen CHEN, Changchun (CN)

(21) Appl. No.: 18/264,346

(22) PCT Filed: Jan. 29, 2022
(86) PCT No.: PCT/CN2022/075135

§ 371 Date: Aug. 4, 2023

(30) Foreign Application Priority Data
Feb. 8, 2021 (CN) 202110186583.1
Mar. 17, 2021 (CN) 202110288214.3

(51) Int. Cl. A61K 31/44; A61P 35/00; A61K 9/00; C07K 16/28

(52) U.S. Cl. CPC A61K 31/44; A61P 35/00; A61K 9/0019; C07K 16/2878; C07K 16/2818

(57) ABSTRACT

The present invention relates to a pharmaceutical use of a PPARS inhibitor in combination with an immunotherapeutic drug for preparing an anti-tumor drug, wherein the immunotherapeutic drug is an immune agonist or an immune checkpoint inhibitor, the tumor is preferably melanoma, mammary cancer, ovarian cancer, pancreatic cancer, lung cancer, liver cancer, esophageal cancer, colorectal cancer, colonic cancer, lymphoma, brain tumor, sarcoma, cervical cancer, prostate cancer, bladder cancer, osteosarcoma, head and neck cancers, renal cell carcinoma, or stomach cancer. The medicine of the present invention exhibits significant anti-cancer effect, strong targeting ability which has little side effect.

This application is a national stage application of International Patent Application No. PCT/CN2022/075135, filed Jan. 29, 2022, which claims priority to CN Application No. 202110186583.1, filed on Feb. 8, 2021, and CN Application No. 202110288214.3, filed on Mar. 17, 2021, which is incorporated in its entirety herein by reference.

### FIELD

The present disclosure relates to technical field of biological medicine, in particular to use of PPARS inhibitor in combination with immunotherapeutic drug for preparation of anti-tumor drug and the anti-tumor drug composition thereof.

### DESCRIPTION OF RELATED ART

In recent years, tumors have become a major threat to human health. In addition to traditional surgery, radiotherapy, and chemotherapy, immunotherapy as an alternative treatment can mobilize the immune system in an organism to specifically skill tumors, and has received more and more attention for its smaller side effects, and better and more lasting therapeutic effects as compared to the traditional therapies. Immunotherapy can be used solely or in combination with radiotherapy or chemotherapy. In addition, different kinds of immunotherapies may be used together to enhance their effects on treating tumors.

The aim of immunotherapy is to build new or facilitate existing anti-tumor immune responses by stimulating the immune system to specifically eliminate tumor cells and generate immunological memory responses specific to the tumor. There are also various ways for tumors to dodge attacks from the immune system, in which an important point is to use the co-suppression mechanism to enable immunologic escape through immunosuppressive molecules such as CTLA-4, and PD-(L) 1 (Beatty G L, Gladney W L. Clin Cancer Res. 2015; 21(4): 687-692.). The first-generation immune checkpoint inhibitors specific to these targets have made significant progress on tumor treatment. Therein, anti-PD-(L) 1 monoclonal antibody (mAb) and anti-CTLA-4 mAb have been approved for treating tens of cancers, but the total efficacy rates are merely about 20%. A prerequisite for these therapies to be effective is that the immune system generates sufficient immune responses against tumor antigen in advance, like the case of hot tumors. Although these tumor patients have T cells that have immune responses to tumors, the functions of these T cells are inhibited by PD-1 signals and CTLA4. Disinhibition works with patients with this kind of tumors. As to cold tumors, or tumors to whose antigen the immune system does not respond sufficiently, it is important to activate the immune system effectively for its efficient work.

Activating costimulatory signals is an important means to stimulate the immune system, and stimulating the path of CD40 costimulatory signals located on antigen presenting cells (APCs) is an effective way to stimulate the immune system. CD40 molecules are mainly expressed on APC cells, including some monocytes, macrophages, and dendritic cells (DCs), and may alternatively be expressed on B cells, platelets, endothelial cells, and smooth muscle cells. CD40L is expressed on activated CD4 T cells, memory CD8 T cells, and activated NK cells. CD40 stimulants promote expression of DC cell costimulatory molecules CD80 and CD86, and major histocompatibility molecules, and also promote release of cytokines having immune-stimulation, thereby enhancing the antigen-presenting function of APC, and exhibit the ability to promote T cell immunity in a tumor model (French R R, Chan H T, Tutt A L, Glennie M J. Nat Med. 1999; 5(5): 548-553.; Sotomayor E M, Borrello I, Tubb E, et al. Nat Med. 1999; 5(7): 780-787.; Diehl L, den Boer A T, Schoenberger S P, et al. Nat Med. 1999; 5(7): 774-779.). Recombinant human CD40L has exhibited preliminary effects in treating advanced physical tumors and non-Hodgkin lymphoma (Vonderheide R H, Dutcher J P, Anderson J E, et al. J Clin Oncol. 2001; 19(13): 3280-3287.), and adenovirus containing CD40L DNA in bladder cancer (Malmstrom P U, Loskog A S, Lindqvist C A, et al. Clin Cancer Res. 2010; 16(12): 3279-3287.), as demonstrated in clinical trials. It can effectively stimulate anti-tumor immune responses of CD8 T cells. As found in a clinical trial where an anti-CD40 agonistic antibody was used to treat pancreatic cancer, CD40 signal can further activate immunologic surveillance of macrophages, making tumor-infiltrating macrophage functionally shift from pro-tumor to anti-tumor (Beatty G L, Chiorean E G, Fishman M P, et al. Science. 2011; 331(6024): 1612-1616.).

While several anti-CD40 agonistic antibodies have clinical shown preliminary therapeutic effects, when used solely, their efficacy is limited, with an overall response rate of merely 14% or so. In view of this, it has been considered to combine an anti-CD40 agonistic antibody and other immunostimulants in use for better immune effects. Immunostimulants for combined used mainly include Type I interferons, IL-2, and TLR receptor antagonists (U.S. Pat. No. 9,095,608B2; Bouchlaka M N, Sckisel G D, Chen M, et al. The Journal of experimental medicine. 2013; 210(11): 2223-2237.; U.S. U.S. Pat. No. 7,993,659B2.). Another key factor that limits clinical applications of anti-CD40 agonistic antibodies is its adverse side effects. Its clinically effective dose is close to its toxic dose, and the side effects mainly include dose-dependent cytokine release syndromes (Vonderheide R H, Flaherty K T, Khalil M, et al. J Clin Oncol. 2007; 25(7): 876-883.) and hepatotoxicity (such as increased transaminase, liver cell necrosis, etc.) (Medina-Echeverz J, Ma C, Duffy A G, et al. Cancer immunology research. 2015; 3(5): 557-566.). According to clinical trials, when administered with IL-2, anti-CD40 agonistic antibodies could cause fatal hepatotoxicity increased with age and pulmonary and intestinal toxicity (Bouchlaka M N, Sckisel G D, Chen M, et al. The Journal of experimental medicine. 2013; 210(11): 2223-2237.). As found in a mouse model, the combination of an anti-CD40 agonistic antibody and chemotherapy drugs even causes lethal toxicity (Long K B, Gladney W L, Tooker G M, et al. Cancer Discov. 2016; 6(4): 400-413.). It is thus clear that a relatively high dose of an anti-CD40 agonist or a combined use of it with other immunostimulants would cause significant side effects due to the overall activation of the immune system, which limit clinical applications of anti-CD40 agonists. Hence, how to reduce the toxicity incurred by administration of high-dose CD40 agonists without affecting or even increasing anti-tumor activity of the drug is a pressing issue to be address before clinical anti-tumor applications of CD40 agonists can be further extended.

Peroxisome proliferators-activated receptors (PPARs) are ligand activated receptors in the family of nuclear hormone receptors. Its three subtypes have been found in different species as controlling factors of many intracellular metabolism processes. The three subtypes of PPARs, namely α, γ, and δ, form a subfamily of nuclear receptors. This PPAR family can usually be activated by endogenous fatty acid (HE Xu, M H Lambert, V G Montana, et al. Molecular cell. 1999; 3(3): 397-403.), and, through transcription of activated target genes (R M Evans, G D Barish, Y X Wang. Nature medicine. 2004; 10(4): 355-361.), control generalized metabolism for fatty acid. PPARγ or PPARδ is essential for maturation of M2 macrophages. Blocking paths for PPARγ or PPARδ signals can lead to polarization of macrophages into their M1 state (J I Odegaard, R R Ricardo-Gonzalez, M H Goforth, et al. Nature. 2007; 447(7148): 1116-20.; J I Odegaard, R R Ricardo-Gonzalez, ARed Eagle, et al. Cell Metab. 2008; 7(6): 496-507.). In the tumor microenvironment, macrophages form a major group of infiltrating leukocytes. They have M2-like phenotype (an inhibited phenotype), promoting progress of tumors and drug resistance in immunotherapy or chemotherapy. Polarizing and reversing them to the M1 state is regarded as an important anti-cancer immunotherapy strategy (D Saha, R L Martuza, S D Rabkin. Cancer Cell. 2017; 32(2): 253-267.e255.; D G DeNardo, B Ruffell. Nat Rev Immunol. 2019; 19(6): 369-382.).

In addition, on one hand, there are differences in the understanding of the technology in the field; on the other hand, although the inventors have studied a large number of documents and patents when making the present invention, due to the limitation of length, all the details and contents are not listed in detail, but this is by no means that the present invention does not possess the features of the prior art, on the contrary, the present invention already possesses all the features of the prior art, and the applicant reserves the right to add relevant prior art to the background.

### SUMMARY OF THE INVENTION

According to the knowledge of the prior art as described above, researchers proposing the present invention assumed that PPARγ or PPARδ inhibitors can enhance anti-CD40 agonistic antibodies in terms of therapeutic effect. The applicant found in studies that when an anti-CD40 agonistic antibody and a PPARδ inhibitor are used in combination, better therapeutic effects on melanoma can be provided as compared to an anti-CD40 agonistic antibody used solely. The combined therapy showed an efficacy rate of 100%. Meanwhile, the combined therapy of an anti-CD40 agonistic antibody and a PPARδ inhibitor has a border range of effective doses than use of an anti-CD40 agonistic antibody solely. In combined use of an anti-CD40 agonistic antibody and a PPARδ inhibitor, low and high doses of the anti-CD40 agonistic antibody showed equivalent, good therapeutic effects on tumors, and a low dose of the anti-CD40 agonistic antibody used in the combination generated almost no hepatotoxicity. Surprisingly, in a melanoma model, only a combination of an anti-CD40 agonistic antibody and a PPARδ inhibitor showed improved therapeutic effects, yet when the same dose of the anti-CD40 agonistic antibody was used with a PPARγ inhibitor, no synergistic anti-tumor effects exhibited, and use of the PPARδ inhibitor solely provided no therapeutic effects.

Based on the applicant's findings described above, the applicant claims the following technical solution:

In a first aspect of the present invention, there is provided a pharmaceutical use of a PPARδ inhibitor in combination with an immunotherapeutic drug in an anti-tumor drug,

the tumor is preferably melanoma, mammary cancer, ovarian cancer, pancreatic cancer, lung cancer, liver cancer, esophageal cancer, colorectal cancer, colonic cancer, lymphoma, brain tumor, sarcoma, cervical cancer, prostate cancer, bladder cancer, osteosarcoma, head and neck cancers, renal cell carcinoma, or stomach cancer.

In the pharmaceutical use above, the immunotherapeutic drug is an immune agonist or an immune checkpoint inhibitor;

preferably the immune agonist is an agonist specific for costimulatory molecules, which include OX40, 4-1BB(CD137), CD27, GITR, CD28, and/or ICOS;

preferably the immune agonist is a CD40 agonist;

preferably the immune checkpoint inhibitor is selected from a PD-1 inhibitor, a PDL1 inhibitor, a TIM3 inhibitor, a LAG3 inhibitor, a CD47 inhibitor; and preferably the immune checkpoint inhibitor is selected from an anti-PD-1 antibody, an anti-PDL1 antibody, an anti-TIM3 antibody, an anti-LAG3 antibody, an anti-CD47 antibody, and an anti-CTLA-4 antibody; and

preferably the immune checkpoint inhibitor is an anti-PD-1 antibody.

In the pharmaceutical use above, the CD40 agonist is selected from an anti-CD40 agonistic antibody, a CD40L protein, an expression vector of a CD40L protein, or a fragment, a derivative, and/or a polymer thereof;

preferably the CD40L protein is a recombinant CD40L protein; and

preferably the CD40 agonist is an anti-CD40 agonistic antibody.

In the pharmaceutical use above, the PPARδ inhibitor is a compound being able to inhibit PPARδ, or a nucleic acid molecule being able to inhibit effects of mRNA of PPARδ, or a molecule being able to decompose PPARδ in a targeted manner;

preferably the nucleic acid molecule is siRNA or shRNA; and

preferably the PPARδ inhibitor is GSK3787.

A second aspect of the present invention provides an anti-tumor drug composition, comprising a PPARδ inhibitor and an immunotherapeutic drug that are prepared separately and then packaged together, or a formulation prepared by mixing the PPARδ inhibitor and the immunotherapeutic drug together;

the tumor is preferably melanoma, mammary cancer, ovarian cancer, pancreatic cancer, lung cancer, liver cancer, esophageal cancer, colorectal cancer, colonic cancer, lymphoma, brain tumor, sarcoma, cervical cancer, prostate cancer, bladder cancer, osteosarcoma, head and neck cancers, renal cell carcinoma, or stomach cancer;

the tumor is preferably melanoma, bladder cancer, or non-small cell lung cancer (NSCLC).

Preferably, the PPARδ inhibitor is a compound being able to inhibit PPARδ, or a nucleic acid molecule being able to inhibit effects of mRNA of PPARδ, or a molecule being able to decompose PPARδ in a targeted manner;

preferably the nucleic acid molecule is siRNA or shRNA; and preferably the PPARδ inhibitor is GSK3787.

Preferably, the immunotherapeutic drug is an immune agonist or an immune checkpoint inhibitor;

further preferably the immune agonist is an agonist specific for costimulatory molecules, which include OX40, 4-1BB(CD137), CD27, GITR, CD28, and/or ICOS;

further preferably the immune agonist is a CD40 agonist;

further preferably the immune checkpoint inhibitor is selected from a PD-1 inhibitor, a PDL1 inhibitor, a TIM3 inhibitor, a LAG3 inhibitor, a CD47 inhibitor;

further preferably the immune checkpoint inhibitor is selected from an anti-PD-1 antibody, an anti-PDL1 antibody, an anti-TIM3 antibody, an anti-LAG3 antibody, an anti-CD47 antibody, and an anti-CTLA-4 antibody.

The anti-tumor drug composition above further comprises a pharmaceutically acceptable carrier, and being prepared into a pharmaceutically acceptable formulation;

preferably the formulation is an injection, a targeting formulation, or a nano-formulation.

A further aspect of the present invention provides a use of a PPARδ inhibitor in combination with an immunotherapeutic drug for treating tumors, comprising, during a course of treatment, administering a low dose of the immunotherapeutic drug to a patient, while administering the PPARδ inhibitor, so as to enhance immunotherapeutic effects, to reduce side effects of the immunotherapeutic drug, or to prevent increase in the dose of the immunotherapeutic drug;

the immunotherapeutic drug is an immune agonist or an immune checkpoint inhibitor;

the PPARδ inhibitor is a compound being able to inhibit PPARδ, or a nucleic acid molecule being able to inhibit effects of mRNA of PPARδ, or a molecule being able to decompose PPARδ in a targeted manner;

preferably the nucleic acid molecule is siRNA or shRNA; and preferably the PPARδ inhibitor is GSK3787.

The immune agonist is an agonist specific for costimulatory molecules, which include OX40, 4-1BB(CD137), CD27, GITR, CD28, and/or ICOS;

preferably the immune agonist is a CD40 agonist;

the immune checkpoint inhibitor is selected from a PD-1 inhibitor, a PDL1 inhibitor, a TIM3 inhibitor, a LAG3 inhibitor, a CD47 inhibitor; and preferably the immune checkpoint inhibitor is selected from an anti-PD-1 antibody, an anti-PDL1 antibody, an anti-TIM3 antibody, an anti-LAG3 antibody, an anti-CD47 antibody, and an anti-CTLA-4 antibody; and

preferably the immune checkpoint inhibitor is an anti-PD-1 antibody.

B cells have two roles in development of tumors. As demonstrated in a large number of researches, B cells and plasma cells may promote growth of tumors in a microenvironment, and this has inspired treatment of physical tumors by means of eradicating B cells. However, some studies made on human tumors found that, different from the pro-tumor effects of B cells provided in a mice tumor model, B cells in human tumors may form tertiary lymphoid structures (TLSs). B cells in a TLS may promote responses to immunotherapy through be promoting presenting tumor antigens to CD4 T cells, and this relates to good prognosis in patients (F Petitprez, A Reynies, E Z Keung, et al. Nature. 2020; 577(7791): 556-560.; B A Helmink, S M Reddy, J Gao, et al. Nature. 2020; 577(7791): 549-555.; R Cabrita, M Lauss, A Sanna, et al. Nature. 2020; 577(7791): 561-565.). Thus, apparently, eradication of B cells can cause harmful results in most patients. Decisions related to use of B cell-targeted therapies and its combined use with other therapies shall be made on the basis of better understanding of the general nature of tumor-immunity interaction mediated by different B cell subpopulations. The reason why B cells performed differently in human-tumor and mouse-tumor models may be the fact that B cells inherently represent a heterogeneous group with various effects, and the mouse-model experiments in the foregoing study were designed to investigate into the role B cells play in occurrence and development of tumors by eradicating the entire B cell group. Therefore, targeting B cells having immunosuppressive effects is an attractive strategy.

Accordingly, in the present invention, the PPARδ inhibitor only targets CD19⁺CD24^{hi}IgD^{lo/-} B cells that have immunosuppressive effects, while does not affect CD19⁺CD24^{lo}IgD^{hi} B cells that stimulate the immune system. This prevents the adverse effects caused by total eradication of B cells. Additionally, the therapeutic strategy of using a PPARδ inhibitor and an immunotherapeutic drug in combination not only increase the effects of immunotherapy but also provides improved safety by preventing patients from being exposed to ineffective immunotherapy and potential serious toxic side effects brought about by immunotherapy.

According to the results of our study, in a combined therapy model of a PPARδ inhibitor and an immunotherapeutic drug, it seems that the PPARδ inhibitor provided therapeutic effects through a mechanism other than affecting polarization of tumor-related macrophages. During assessment of the combined therapeutic mechanism of the anti-CD40 agonistic antibody and the PPARδ inhibitor on melanoma, it was found that the number and proportion of T cell-suppressing B cells in draining lymph nodes increased significantly. Eradicating B cells or suppressing the immunosuppressive effects of B cells using the PPARδ inhibitor significantly enhanced the therapeutic effects of the low-dose anti-CD40 agonistic antibody. Besides, eradication of all B cells or use of the PPARδ inhibitor also helped improve the anti-tumor effects of the anti-PD −1 antibody. Further researches suggest that a tumor itself can induce increase of CD19⁺CD24^{hi}IgD^{lo/-} B cells in draining lymph nodes. By comparison, CD19⁺CD24^{hi}IgD^{lo/-} B cells express more PPARδ, and have stronger proliferous ability and immunosuppressive effects, while CD19⁺CD24^{hi}IgD^{hi} B cells have better immune-stimulation. The PPARδ inhibitor can reduce the proliferous and immunosuppressive effects of CD19⁺CD24^{hi}IgD^{lo/-} B cells, and its side effects are minute, thus being safe.

Another aspect of the present application relates to a method for treating tumor. The method comprises the steps of administering to a subject in need of such treatment an effective amount of (1) a PPARδ inhibitor and (2) an immune stimulator or immune checkpoint inhibitor, wherein (1) and (2) may be administered simultaneously or separately. In some embodiments, the tumor is melanoma, bladder cancer, or non-small cell lung cancer (NSCLC).

In some embodiments, the PPARδ inhibitor comprises a small molecule inhibitor of PPARS.

In some embodiments, the PPARδ inhibitor comprises siRNA or srRNA or shRNA. In some embodiments, the PPARδ inhibitor comprises GSK3787.

In some embodiments, the PPARδ inhibitor is administered daily for a period of 1-14 days, or every 2, 3, 4, 5, 6, 7, 8, 9, or 10 days for a period of 2-30 days, or every 2, 3 or 4 weeks for a period of 2-24 weeks. The PPARδ inhibitor may be administered orally, intramuscularly, intravenously, or intraperitoneally.

In some embodiments, the PPARδ inhibitor is GSK3787 and is administered at a unit dose of 1-100 mM/kg body weight, 1-30 mM/kg body weight, 1-10 mM/kg body weight, 1-3 mM/kg body weight is GSK3787 and is administered at a unit dose of 1-100 mM/kg body weight, 1-30 mM/kg body weight, 1-10 mM/kg body weight, 1-3 mM/kg body weight, 3-100 mM/kg body weight, 3-30 mM/kg body weight, 3-10 mM/kg body weight, 10-100 mM/kg body weight, 10-30 mM/kg body weight, or 30-100 mM/kg body weight.

In some embodiments, the immune stimulator or immune checkpoint inhibitor is a CD40 stimulator. In some embodiment, the CD40 stimulator is an anti-CD40 antibody.

In some embodiments, the immune stimulator or immune checkpoint inhibitor is administered daily for a period of 1-14 days, or every 2, 3, 4, 5, 6, 7, 8, 9, or 10 days for a period of 2-30 days, or every 2, 3 or 4 weeks for a period of 2-24 weeks. The immune stimulator or immune checkpoint inhibitor may be administered orally, intramuscularly, intravenously, or intraperitoneally.

In some embodiments, the immune stimulator is an anti-CD40 antibody and is administered at a unit dose of 0.1-30 mg/kg body weight, 0.1-10 mg/kg body weight, 0.1-3 mg/kg body weight, 0.1-1 mg/kg body weight, 0.1-0.3 mg/kg body weight, 0.3-30 mg/kg body weight, 0.3-10 mg/kg body weight, 0.3-3 mg/kg body weight, 0.3-1 mg/kg body weight, 1-30 mg/kg body weight, 1-10 mg/kg body weight, 1-3 mg/kg body weight, 3-30 mg/kg body weight, 3-10 mg/kg body weight, or 10-30 mg/kg body weight.

The present invention at least has the following technical benefits:
1) The PPARδ inhibitor can reduce the proliferous and suppressive effects of tumor-induced CD19⁺CD24^{hi}IgD^{lo/-} B cells, and can be used in combination with a CD40 agonist for anti-tumor treatment. The present invention enhances the ability of the CD40 agonist to stimulate immune responses while reducing the required amount of the CD40 agonist, and providing significantly improved tumor-curing effects of the enhance CD40 agonist, thereby preventing toxic side effects of the CD40 agonist used in a high dose.
2) The present invention further proves that when administered to tumor-bearing mice, the combination of the PPARδ inhibitor and the low-dose CD40 agonist effectively excited the treated organisms to generate anti-tumor T cells, and elicited memory responses of the anti-tumor T cells, so as to significantly enhance the anti-tumor effects of anti-tumor T cells.
3) The present invention proves that the PPARδ inhibitor can be alternatively used in combination with a different immunotherapeutic drug (an immune agonist or an immune checkpoint inhibitor) for anti-tumor treatment.
4) As compared to CD19⁺CD24^{lo}IgD^{hi} B cells, CD19⁺CD24^{hi}IgD^{lo/-} B cells express higher PPARS. In the combined therapy model of the PPARδ inhibitor and the immunotherapeutic drug of the present invention, the PPARδ inhibitor only reduces the proliferous and suppressive effects of immune-suppressing CD19⁺CD24^{hi}IgD^{lo/-} B cells, and affects immune-stimulating CD19⁺CD24^{lo}IgD^{hi} B cells very little. This prevents harmful results in most patients that might otherwise be caused by exhaustion of B cells, making the combined therapy highly target specific and very safe as its side effects are minute.
5) The combination therapy of the present disclosure allows the use of low dose anti-CD40 antibody to reduce liver toxicity.

### BRIEF DESCRIPTION OF THE DRAWINGS

FIG. 1 graphically shows dose-dependent anti-tumor effects of FGK45.5 as obtained in an experiment in Example 1 of the present invention;

FIG. 2 graphically shows therapeutic results of the combination of a PPARδ inhibitor and FGK45.5 obtained in a mouse experiment as obtained in Example 2 of the present invention;

FIG. 3 represents immune effects of the combined therapy of FGK45.5 and a PPARδ inhibitor as obtained in Example 3 of the present invention;

FIG. 4 graphically shows subpopulations and phenotypic variations of lymphocytes in draining lymph nodes in tumor-bearing mice of different treated groups as obtained in Example 4 of the present invention;

FIG. 5 graphically shows experimental results proving that the PPARδ inhibitor reduced immunosuppressive effects of B cells as obtained in Example 5 of the present invention;

FIG. 6 graphically shows therapeutic effects of the combined therapy of PPARδ inhibitor and the anti-PD-1 antibody on B16 tumor-bearing mice as obtained in Example 6 of the present invention;

FIG. 7 graphically shows experimental results of the PPARδ inhibitor reducing proliferation of CD19⁺CD24^{hi}IgD^{lo/-} B cells as obtained in Example 7 of the present invention; and

FIG. 8 graphically shows experimental results of the PPARδ inhibitor reducing suppressive effects of CD19⁺CD24^{hi}IgD^{lo/-} B cells as obtained in Example 8 of the present invention;

FIG. 9 graphically shows the PPARδ inhibitor enhancing effects of FGK45.5 in treating mouse MB49 tumor;

FIG. 10 graphically shows the PPARδ inhibitor reducing immunosuppressive effects of CD19⁺CD24^{hi}IgD^{lo/-} B cells from non-small cell lung cancer (NSCLC) patients.

In the graphs, Rat IgG represents the control antibody group, FGK represents the anti-CD40 agonistic antibody FGK45.5, T007 represents the PPARγ inhibitor T0070907, GSK represents the PPARδ inhibitor GSK3787, Naive B represents B cells from natural untreated mice, No B represents the group where no B cells are added into the co-culture system, Naive represents the group of natural mice, and NS represents no statistical significance, *P<0.05, **P<0.01, ***P<0.001, ****P<0.0001.

### DETAILED DESCRIPTION OF THE INVENTION

The following detailed description is given in conjunction with the accompanying drawings.

The present invention will be further described below in conjunction with the examples, but the present invention is not limited thereby.

The experimental methods in the following examples are conventional methods unless otherwise specified. The materials, reagents, etc. used in the following examples can be obtained through commercial channels unless otherwise specified.

Main sources of reagents:
PPARγ inhibitor T0070907, purchased from Selleck Chemicals, USA;
PPARδ inhibitor GSK3787, purchased from Selleck Chemicals, USA;
FGK45.5 (mouse anti-CD40 agonistic antibody): purchased from BioXcell, USA (West Lebanon, NH);
Rat IgG: Clone No. 2A3, purchased from BioXcell, USA (West Lebanon, NH);
Anti-CD19 antibody: Clone No. 1d3, purchased from BioXcell, USA (West Lebanon, NH);
Anti-PD −1 antibody: Clone No. RMP1-14, purchased from BioXcell, USA (West Lebanon, NH);
anti-CD3ε antibody: Clone No. 145-2C11, purchased from Biolegend, USA (San Diego, CA);
Anti-CD28 antibody: Clone No. 37.51, purchased from Biolegend, USA (San Diego, CA);
B16 tumor cells: purchased from ATCC, USA (the American Type Culture Collection) cell bank.

### Example 1: Anti-CD40 Agonistic Antibody Showed Dose-Dependent Anti-Tumor Effects, but High Doses of which could Incur Obvious Hepatotoxicity

The anti-CD40 agonistic antibody can promote shift from cold tumors to hot tumors, and has medium anti-tumor effects on some tumors, yet it has a narrow range of effective doses, and is toxic to liver and some other tissues. As reported, the toxic effects can be mitigated by intra-tissue injection and slow release of the anti-CD40 agonistic antibody in the tumor drainage area.

First, we tested whether peritumoral injection of the anti-CD40 agonistic antibody incurs obvious toxicity.

We used a mouse B16 melanoma model extensively used in researches and having relatively high immunogenicity (C57BL/6 B16 Tumor-Bearing Mouse Model): C57BL/6 mice (8-10-week-old, n=5/group) were subcutaneously inoculated with 7×10⁵ B16 tumor cells (mouse melanoma cells), and about 8 days after inoculation of tumor cells when tumors became touchable, anti-CD40 agonistic antibody was administered.

The mice were monitored every day, and tumors were measured with calipers every four days. The tumors were measured for length (a) and width (b), and tumor volume=ab²/2.

Dosage regimen: These tumor-bearing mice were treated using low-dose 25 μg of FGK45.5 (mouse anti-CD40 agonistic antibody) for three times, 75 μg in total (at Day 8, Day 11, and Day 14) or high-dose 40 μg for 5 times, 200 μg in total (at Day 8, Day 11, Day 14, Day 17, and Day 20) or 40 μg with Isotype Control Rat IgG for 5 times (at Day 8, Day 11, Day 14, Day 17, and Day 20). Subcutaneous injection was made between the tumor and the draining lymph node at the groin. 72 hours after the last injection of each group, samples of mouse blood serum were tested for alanine transaminase (ALT, a known indicator of liver tissue injury).

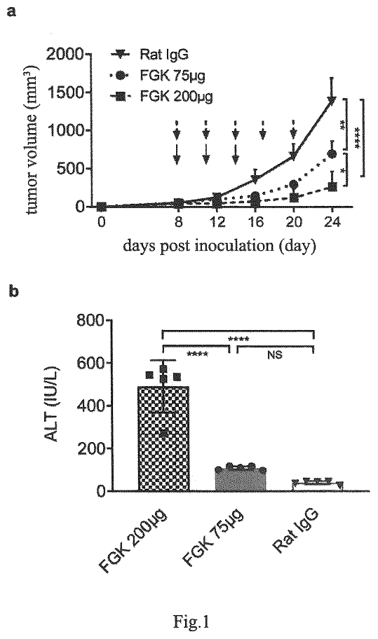

The results are shown in FIG. 1a. The mice injected with FGK45.5 had lower tumor load than the control, but the high-dose FGK45.5 group exhibited more obvious anti-tumor effects.

The results indicate that anti-tumor effects of FGK45.5 were dose-dependent.

ALT was analyzed for assessment of toxicity of the treatments. Mice in the low-dose FGK45.5 group had less liver injury, and the blood serum ALT was about 100 units, slightly higher than the control at about 40 units. It is to be noted that the high-dose FGK45.5 group had further increased toxicity. The mice treated with high-dose FGK45.5 showed more serious liver injury, with the ALT level significantly increased to about 500 units (FIG. 1b).

These data indicate that higher doses of the agonistic CD40 antibody administrated through local subcutaneous injection helped enhance anti-tumor effects in this therapy, yet they incurred relatively serious hepatotoxicity. Therefore, the strategy of enhancing anti-tumor effects of anti-CD40 agonistic antibody through increasing its dose is infeasible. Therefore, how to reduce toxic side effects of agonistic CD40 antibody while ensuring its anti-tumor effects is crucial to extending its clinical applications.

### Example 2: PPARδ Inhibitor Enhanced Therapeutic Effects of Low-Dose Anti-CD40 Agonistic Antibody without Incurring Obvious Hepatotoxicity, and PPARγ Inhibitor Provided No Such Effects

As reported, PPARγ or PPARδ is a sensor of fatty acid, and is essential for maturation of M2 (an inhibited phenotype) macrophages. Blocking the path of PPARγ or PPARδ leads to polarization of macrophages to their M1 state. In a tumor microenvironment, macrophages form a major group of infiltrating lymphocytes, having M2-like phenotype, and promoting progress of tumors and drug resistance in immunotherapy or chemotherapy. Polarizing and reversing these cells to the M1 state is regarded as an important anti-cancer immunotherapy strategy. In view of the ability to turn tumor-associated macrophages (TAMs) into the major M1 phenotype, we used the B16 tumor-bearing mouse model of Example 1 and the low-dose FGK45.5 regimen to test whether the PPARγ inhibitor (T0070907, Selleck, USA) or the PPARδ inhibitor (GSK3787, Selleck, USA) and the anti-CD40 agonistic antibody have combined anti-tumor effects.

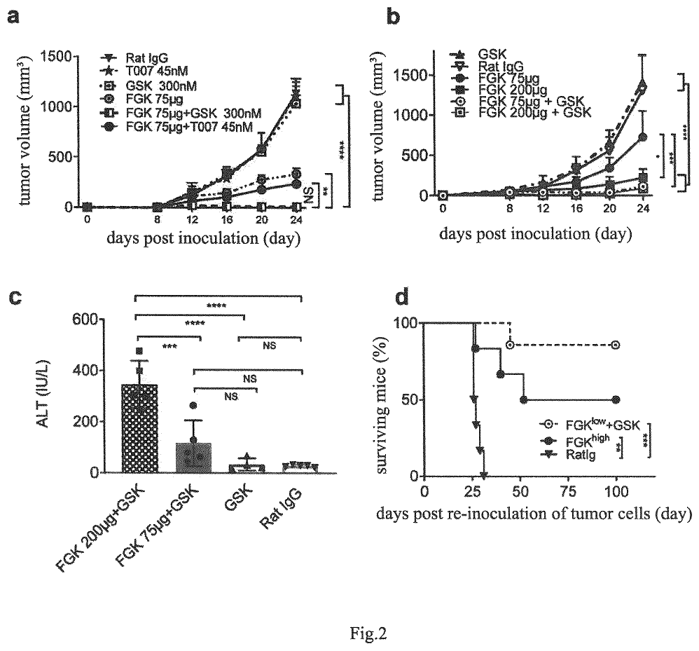

The establishment of the B16 melanoma tumor-bearing mouse model and the dosage regimens of low-dose and high-dose FGK45.5 followed Example 1. The combined group of low-dose FGK45.5 with T0070907 or GSK3787 was administrated, at Day 7 to Day 16 after injection of B16 cells, T0070907 or GSK3787 with the doses marked in FIGS. 2a, 2b through intraperitoneal injection, once a day. The combined group of high-dose FGK45.5 with T0070907 or GSK3787 was administrated, at Day 7 to Day 22 after injection of B16 cells, T0070907 or GSK3787 with the doses marked in FIG. 2b through intraperitoneal injection, once a day. The group of T0070907 or GSK3787 solely was administrated T0070907 or GSK3787 through intraperitoneal injection at Day 7 to Day 16 after injection of B16 cells with doses marked in FIG. 2a, 2b, once a day.

Consistent with previous researches, the use of low-dose FGK45.5 solely (75 μg in total) only exhibited very small anti-tumor effects, and the use of the PPARγ inhibitor or the PPARδ inhibitor solely for treatment provided almost no anti-tumor effects. However, FGK45.5 and the PPARδ inhibitor when used in combination significantly reduced the speed of tumor growth. On the other hand, FGK45.5 and the PPARγ inhibitor did not generate synergistic anti-tumor effects (FIG. 2a).

Then we further examined effects of the PPARδ inhibitor and combined therapies of it with different doses of FGK45.5. The PPARδ inhibitor and low-dose FGK45.5 jointly provided therapeutic effects basically equivalent to those provided by the same inhibitor together with high-dose FGK45.5, and consistent to those of high-dose FGK45.5. The three treatment regimens all showed obvious, almost equivalent anti-tumor effects.

The anti-tumor effects of the combined group of high-dose FGK45.5 and the PPARδ inhibitor, the combined group of low-dose FGK45.5 and the PPARδ inhibitor, and the group of high-dose FGK45.5 solely measured as tumor incidence rates by their tumor growth volume curves are 62% (⅝), 77% ( 6/9), and 87% (⅞), respectively, and the efficacy rates are all 100%, while the tumor incidence rate and efficacy rate of the group of low-dose FGK45.5 solely are 100% (8/8) and 50% ( 4/8), respectively (FIG. 2b).

More importantly, the combined group of low-dose FGK45.5 and the PPARδ inhibitor, when compared to the combined group of high-dose FGK45.5 and the PPARδ inhibitor, had a significantly reduced hepatotoxicity indicator (the ALT level in blood serum). 24 hours after the last injection of the PPARδ inhibitor (GSK3787), mice tail lateral vein blood was sampled for each group. The blood serum was isolated to test the ALT level. The combined group of low-dose FGK45.5 and the PPARδ inhibitor had a blood serum ALT level of about 100IU/L, equivalent to that of the group of mice receiving low-dose FGK45.5 solely, while the combined group of high-dose FGK45.5 and the PPARδ inhibitor had a blood serum ALT level of about 500IU/L. The difference therebetween is statistically significant (FIG. 2c).

Inducing the immune system to generate anti-tumor immunological memory responses is believed to be the key for the immune system to give lasting anti-tumor effects and to prevent tumor recurrence. The experiment described below was conducted to further verify whether the combined use of FGK45.5 and the PPARδ inhibitor is able to induce anti-tumor immunological memory responses in organisms:

In the B16 tumor-bearing mouse model and treatment regimens described above, after completion of the treatment, both the group of combined low-dose FGK45.5 and PPARδ inhibitor and the group of mice receiving high-dose FGK45.5 solely had some tumor-free mice. These tumor-free mice were then used in a memory experiment as described below. Ninety days after the first treatment, 5×10⁵ B16 tumor cells were injected into tail lateral veins of each of the tumor-free mice to excite the immune system again. The control group was composed of age-matching mice not injected with B16 tumor cells.

It is to be noted that 92% of mice in the two treated groups survived more than 40 days, and all mice in the control group died in 31 days. In addition, 80% of the mice in the combined therapy group of low-dose FGK45.5 and the PPARδ inhibitor survived for more than 100 days, yet only 50% of the mice in the group treated with high-dose FGK45.5 solely survived for more than 100 days. However, the difference therebetween has no statistical significance. These results indicate that the combined therapy of low-dose FGK45.5 and the PPARδ inhibitor could similarly induce the immune system to generate anti-tumor memory responses equivalent to those induced using high-dose FGK45.5 solely (FIG. 2d).

The above results suggest that in the group of mice receiving FGK45.5 and the PPARδ inhibitor in combination, the PPARδ inhibitor significantly enhance the therapeutic effects of the low-dose anti-CD40 agonistic antibody without increasing liver injury. The combined therapy of low-dose FGK45.5 and the PPARδ inhibitor achieved both good safety and good efficacy. For the sake of safety, the subsequent experiments were all conducted with FGK45.5 at low doses, and this was applied to all regimens, whether the components were used solely or in combination.

### Example 3: Combined Therapy of FGK45.5 and PPARδ Inhibitor Effectively Increased Infiltration Depth of CD8 T Cells in Tumors

Cells with immune effects in tumors of the group receiving FGK45.5 solely, the group receiving low-dose FGK45.5 and the PPARδ inhibitor in combination, and the control group were quantified using flow cytometry, histologic section, and immunocyte staining.

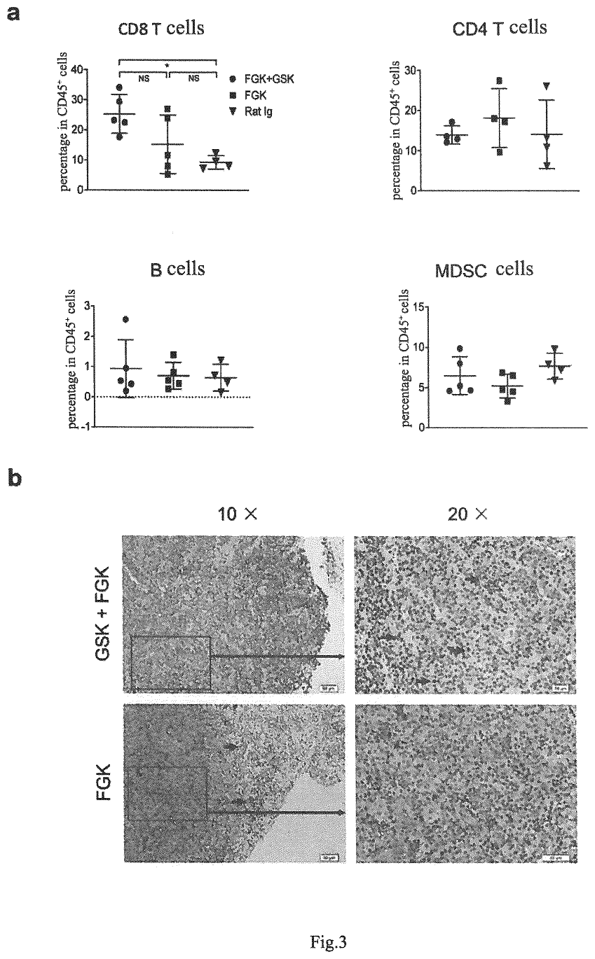

The results are shown in FIG. 3. Although the combined therapy caused the proportion of CD8⁺ T cells in CD45⁺ cells in the treated tumor to only increase little as compared to the treatment using FGK45.5 solely and the difference had no statistical significance, when compared with the control group, the proportion of CD8⁺ cytotoxic T cells in CD45⁺ cells in the tumors treated with the combined therapy increased significantly. In the treatment group of mice receiving FGK45.5 in combination with the PPARδ inhibitor, the proportion of CD8⁺ T cells in CD45⁺ lymphocytes in the tumor increased to about 25%, and that of the treatment group of mice receiving FGK45.5 solely was about 15%, while that of the control group was only 10%. By comparison, the proportions of CD4⁺ T cells, B cells, and myeloid-derived suppressor cells (MDSCs) in CD45*cells showed no significant differences across the treatment groups (FIG. 3a). Interestingly, as found using tumor immunohistochemistry, in the tumors treated with FGK45.5 solely, CD8⁺ T cells mainly existed at the periphery, but in the combined therapy group of FGK45.5 and the PPARδ inhibitor, CD8⁺ T cells infiltrated deeper into the tumors (FIG. 3b).

### Example 4: FGK45.5 Incurred Increase of B Lymphocytes in Draining Lymph Nodes

Activation of T cells caused by antigens happens in lymph nodes, and the variation of cells in lymph nodes of tumor-bearing mice incurred by different treatments should be more obvious than in any other portion. The subsequent experiment was conducted to identify subpopulations and phenotypic variations of lymphocytes at draining lymph nodes in tumor-bearing mice treated with different regimens.

The C57BL/6 B16 tumor-bearing mouse model (mice subcutaneously inoculated with 7×10⁵ B16 cells, 8 days after inoculation of tumor cells, mice with tumors touchable) of Example 1 was used. The experiment included the treatment group of low-dose FGK45.5, the treatment group of low-dose FGK45.5 in combination with the PPARδ inhibitor, the treatment group of using the PPARδ inhibitor solely, and the isotype control Rat IgG treatment group. The treatment regimens of Example 2 shown in FIG. 2a were followed. 24 hours after the last injection of GSK3787 (i.e., 72 hours after the last FGK45.5 injection), the mice of all group were sacrificed. Cells at lymph nodes of mice from all groups were sampled for flow cytometry assays and cell counting.

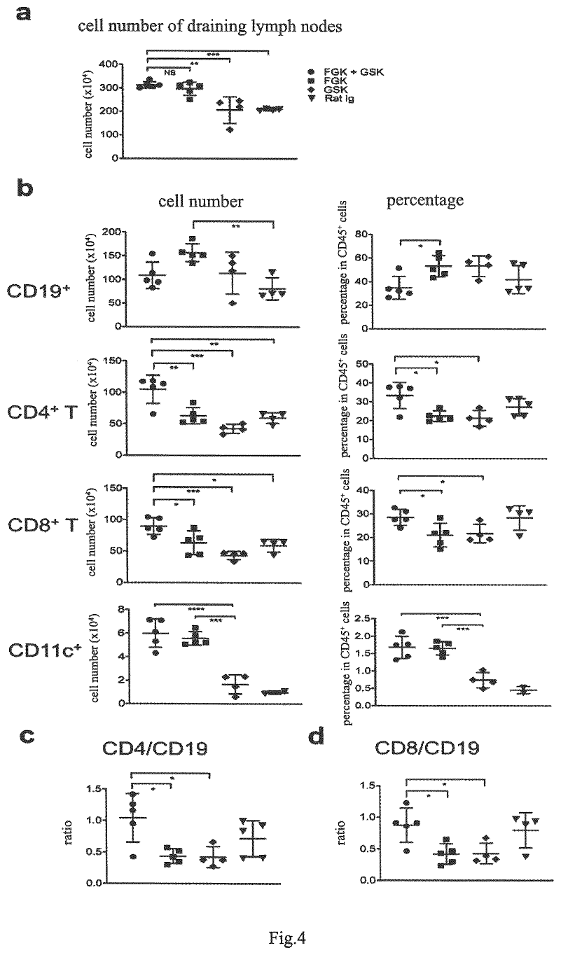

The results are shown in FIG. 4. In the draining lymph nodes, the group of mice receiving low-dose FGK45.5 and the PPARδ inhibitor in combination and the group of mice receiving FGK45.5 solely basically had similar absolute counts of CD45⁺ cells, both significantly higher than those of the PPARS-inhibitor group and the Rat IgG control. The use of the PPARδ inhibitor did not change the absolute count of CD45⁺ cells in the draining lymph nodes (FIG. 4a). Therein, the CD19⁺ cell count of the FGK45.5 group is significantly higher than that of the Rat IgG control, and the CD19⁺ cell count of the group of mice receiving low-dose FGK45.5 and the PPARδ inhibitor in combination is slightly lower than that of the FGK45.5 group. However, the proportion of CD19 cells in CD45⁺ cells in the group of mice receiving low-dose FGK45.5 and the PPARδ inhibitor in combination was significantly lower than that of the FGK45.5 group. The cell counts of CD4⁺ T and CD8⁺ T cells and the proportions in CD45⁺ cells in the group of mice receiving FGK45.5 solely, the group of the PPARδ inhibitor, and the Rat IgG control group are consistent, but in the group of mice receiving low-dose FGK45.5 and the PPARδ inhibitor in combination, both the cell counts of CD4⁺ T and CD8⁺ T cells and their proportions in CD45⁺ cells are significantly higher than those of the group of mice receiving FGK45.5 solely. The absolute count and proportion of CD11c⁺ cells in the group of mice receiving FGK45.5 and the PPARδ inhibitor in combination and in the group of FGK45.5 are basically comparable, and both of the two group had these values significantly higher than those of the PPARδ inhibitor group and the Rat IgG control group. The absolute counts and proportions of CD11c⁺ cells in the PPARδ inhibitor group and the Rat IgG control group are basically the same (FIG. 4b). The proportions of CD4/CD19 cells in the FGK45.5 group, the PPARδ inhibitor group, and the Rat IgG group are basically comparable, but in the group of mice receiving FGK45.5 and the PPARδ inhibitor in combination, the values significantly increased (FIG. 4c). The values of CD8/CD19 in the four experimental groups are similar to the case of CD4/CD19 cells in the four experimental groups (FIG. 4d).

In consideration that M2 macrophages represent an important suppressing factor in a tumor microenvironment, and that the PPARγ or PPARδ inhibitor are reported to be favorable to polarization of macrophages from M2 to M1, we initially assumed that the PPARγ or PPARδ inhibitor enhances the therapeutic effects of the anti-CD40 agonistic antibody through promoting TAM to turn from M2 to M1 after its polarization. However, the results revealed that the combined therapy of FGK45.5 and the PPARδ inhibitor significantly reduced the speed of tumor growth, and the combined therapy of FGK45.5 and the PPARγ inhibitor showed no anti-tumor effects. These results indicate that the PPARδ inhibitor might enhance the therapeutic effects of the anti-CD40 agonistic antibody through a different mechanism instead of driving TAM to turn from M2 to M1 via polarization. As can be seen from the above results, the PPARδ inhibitor when used solely had almost no effects on the counts of CD4 T, CD8 T, CD19⁺ B, and CD11c⁺ cells. FGK45.5 itself alone increased CD19⁺ B and CD11c⁺ cells in lymph nodes. The PPARδ inhibitor suppressed increase in count and proportion of CD19⁺ B cells in the draining lymph nodes caused by FGK45.5, but had no effects on increase in count and proportion of CD11c cells caused by FGK45.5.

The foregoing results indicate that the ability of the PPARδ inhibitor to enhance the anti-tumor effects of low-dose FGK45.5 may be related to its effects on B cells.

### Example 5: PPARδ Inhibitor Reduced Immunosuppressive Effects of B Cells, Thereby Enhancing Immunotherapeutic Effects of FGK45.5

The next point was to verify whether the PPARδ inhibitor enhances immunotherapy through affecting B cells. First, the ability of B cells in draining lymph nodes of tumor-bearing mice treated differently to inhibit proliferation of activated T cells was examined in vitro. Purified C D4⁺ T cells from normal mice were labelled using 2.5 μM CFSE in vitro. The CFSE-labelled cells were then resuspended in complete RPMI 1640 culture medium at 1×10⁶/ml, and transferred into a 96-well plate that had been coated with 10 μg/ml anti-CD3ε antibody at 4° C. overnight. B cells purified from draining lymph nodes and the foregoing T cells (1:1) were co-cultured for 72 hours in the foregoing 96-well plate containing 1 μg/ml anti-CD28 mAb, and a flow cytometry assay was conducted. The cell proliferation rate of the CFSE-labelled T cells was calculated using the equation: (1-MFI value of CFSE of T Cells in Experimental Group/MFI value of CFSE of B-cell-free T Cells in Control Group)×100%. MFI refers to the mean fluorescence intensity.

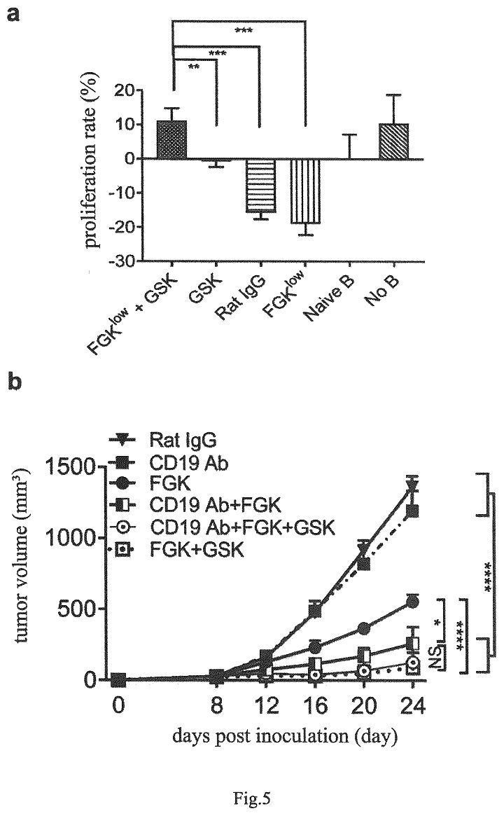

The results reveal that the B cells derived from draining lymph nodes of untreated tumor-bearing mice suppressed CD4 T cells, and the B cells from draining lymph nodes of tumor-bearing mice treated with the PPARδ inhibitor had decreased inhibition. Treating with FGK45.5 did not reduce inhibition of B cells (FIG. 5a). These results indicate that the PPARδ inhibitor might enhance the immunotherapeutic effects of FGK45.5 by weakening the immunosuppressive effects of B cells.

In order to verify the assumption, the antibody was used to eradicate B cells in the B16 tumor-bearing mice to further assess the role of the PPARδ inhibitor in the combined regimen of FGK45.5 and the PPARδ inhibitor.

To some of the B16 tumor-bearing mice, the anti-CD19 antibody was used first to kill B cells, and then Rat IgG or FGK45.5 or FGK45.5 combined with the PPARδ inhibitor (GSK3787) was administered for treatment. Growth of the tumors was observed. Treatment with Rat IgG, FGK45.5, and FGK45.5 in combination with the PPARδ inhibitor were taken as control groups. In these groups, doses and regimens of Rat IgG, FGK45.5 and the PPARδ inhibitor (GSK3787) all followed the doses and regimens of Example 2, as shown in FIG. 2a.

CD19⁺ B cell Eradication Experiment: At the 6^{th} day, 7^{th} day, and 15^{th} day after inoculation of B16 cells, 250 μg anti-CD19 antibody (clone 1d3) was administered to tumor-bearing mice through intraperitoneal injection, and the eradication rate of B cells as measured using FACS should be 90% or more. The experimental results reveal that: tumors in the group of eradicating B cells and the control group of IgG treatment had similar growth condition, meaning that killing B cells solely provided no therapeutic effects on B16 tumor, and killing B cells significantly enhanced therapeutic effects of FGK45.5 on B16 tumors. More importantly, eradication of B cells in combination with FGK45.5 and the FGK45.5-PPARδ group had basically comparable therapeutic effects on B16 tumor. It is more noteworthy that with B cells eradicated, FGK45.5 solely and the FGK45.5-PPARδ group had basically comparable therapeutic effects on B16 tumors, and the PPARδ inhibitor lost its function of enhancing anti-tumor effects of FGK45.5 (FIG. 5b).

These results indicate that in the B16 tumor model, B cells acted as an immunosuppressive factor of FGK45.5 treatment, and the PPARδ inhibitor enhanced anti-tumor effects of low-dose FGK45.5 by suppressing immunosuppressive effects of B cells.

### Example 6: Therapeutic Effects of PPARδ Inhibitor Combined with Anti-PD-1 Antibody on B16 Tumor-Bearing Mice

From the above results, we anticipated that eradication of B cells or use of the PPARδ inhibitor can enhance effects of other immunotherapeutic drug in the B16 tumor-bearing mouse model.

In order to verify this possibility, the PPARδ inhibitor or eradication of B cells was used with the anti-PD −1 antibody to treat B16 tumor-bearing mice and assess the respective therapeutic effects.

Tumor-bearing mice of the same condition were treated using anti-PD −1 antibody, B cell eradication+ anti-PD −1 antibody, anti-PD−1 antibody+ PPARδ inhibitor (7 to 16 days after injection of B16 cells, GSK3787 at doses shown in FIG. 2a (300 nmol), intraperitoneal injection, once a day), respectively. Eradication of B cells (e.g., the “CD19⁺ B Cell Eradication Experiment” of Example 5) and the IgG-treated group (isotype control of rat IgG injection with regimens of Example 1) were used as the control groups, 5 parallel groups (n=8/group) in total.

The anti-PD−1 antibody was used in the following way: from the 8^{th} day after inoculation of tumor cells, the anti-PD−1 antibody (clone RMP1-14) was administered to tumor-bearing mice through intraperitoneal injection, 200 μg every time, once in four days, 4 times of injection in total.

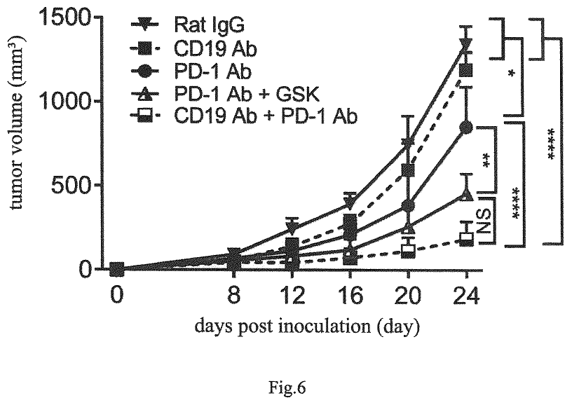

It was found that when used solely, the anti-PD−1 antibody had moderate therapeutic effects on B16 tumors (with a response rate of 4/8), but eradication of B cells or use of the PPARδ inhibitor further enhanced the therapeutic effects of the anti-PD−1 antibody treatment on B16 tumors (with a response rate of ⅞) (FIG. 6).

### Example 7: PPARδ Inhibitor Reduced Proliferation of CD19⁺CD24^{hi}IgD^{lo/-} B Cells

Based on the above results, we assumed that tumor occurrence or FGK45.5 treatment incurs change of B cells in draining lymph nodes and B cells in tumors, making these B cells suppressive to immunotherapy.

For verifying this assumption, the experimental scheme of Example 4 was used. The mice of all groups were sacrificed at the 24^{th} hour after the last injection of GSK3787 (i.e., the 72^{nd} hour after the last FGK45.5 injection), and various surface marker analyses were conducted on the B cells isolated from lymph nodes of tumor-bearing mice to identify surface markers of B cell subpopulations having immunosuppressive effects and the effects of different treatments on B cell subpopulations having immunosuppressive effects.

According to known surface markers of regulatory B cells and key development phenotype, such as CD24, CD38, CD138 or IgD, it was found that CD19⁺CD24^{hi}IgD^{lo/-} B cell populations in different treatment groups changed as below:

At the 24^{th} hour after the last GSK3787 injection, tumor draining lymph nodes of differently treated mice were sampled. The lymph nodes at the same body portion in natural, tumor-free mice were also sampled as the control group. The lymph nodes were ground into single-cell suspension. Cells from different lymph nodes were stained using various fluorescence labeling antibodies for flow cytometry assays.

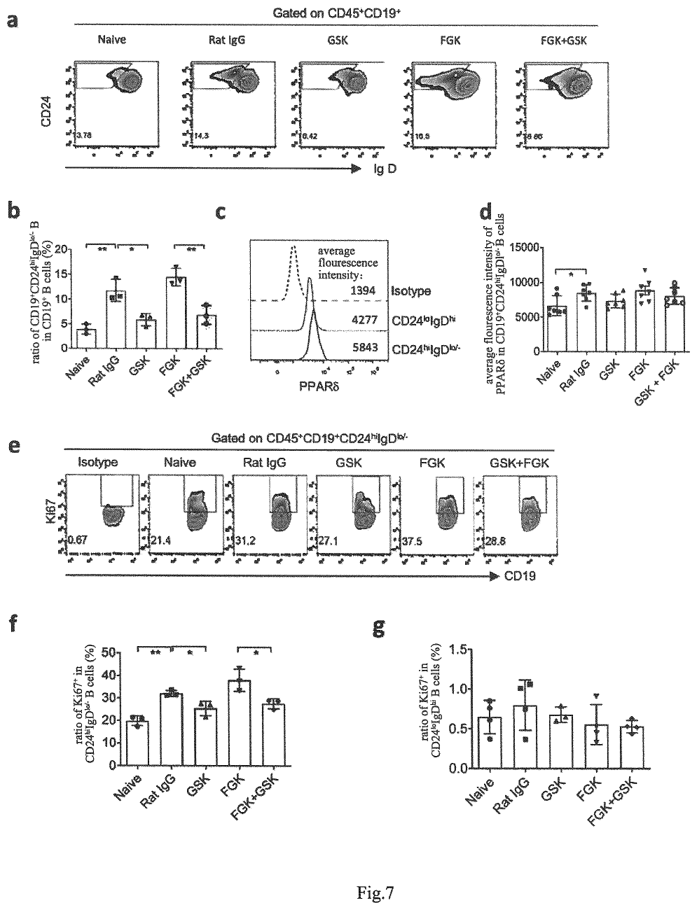

According to the results, in the B cell population, CD19⁺CD24^{hi}IgD^{lo/-} B cells took the smallest proportion in natural mice. In tumor-bearing mice, the proportion of the CD19⁺CD24^{hi}IgD^{lo/-} B cell subpopulation in draining lymph nodes increased significantly. In the FGK treatment group, the proportion of this subpopulation further increased. In the group of mice receiving the PPARδ inhibitor solely, the proportion of this subpopulation of B cells decreased significantly. In the group of using FGK together with the PPARδ inhibitor, the proportion of this subpopulation of B cells was much smaller than that of the FGK treatment group (FIGS. 7a, b). Also, as compared to CD19⁺CD24^{lo}IgD^{hi} B cell population, CD19⁺CD24^{hi}IgD^{lo/-} B cells expressed higher PPARδ (FIG. 7c). The tumor factor promoted PPARδ expression of CD19⁺CD24^{hi}IgD^{lo/-} B cells, but stimulation caused by FGK45.5 did not lead to significant change in PPARδ expression of CD19⁺CD24^{hi}IgD^{lo/-} B cells (FIG. 7d).

In addition, as found in flow cytometry assays, both the tumor factor and FGK promoted Ki67 expression of CD19⁺CD24^{hi}IgD^{lo/-} B cells, and the PPARδ inhibitor reduced increase in Ki67 expression of CD19⁺CD24^{hi}IgD^{lo/-} B cells caused by these two causes (FIG. 7e, f). However, proliferation of CD19⁺CD24^{lo}IgD^{hi} B cell population was not affected by various treatment factors (FIG. 7g).

### Example 8: PPARδ Inhibitor Reduced Suppressive Effects of CD19⁺CD24^{hi}IgD^{lo/-} B Cells

For evaluating the function-regulating effects on CD19⁺CD24^{hi}IgD^{lo/-} B cells, the experimental scheme of Example 4 was employed. 24 hours after the last GSK3787 injection (i.e., 72 hours after the last FGK45.5 injection), mice of all groups were sacrificed. CD19⁺CD24^{hi}IgD^{lo/-} B cell population and CD19⁺CD24^{lo}IgD^{hi}B cell population were isolated form draining lymph nodes of differently treated tumor-bearing mice using flow cytometry. Purified CD4⁺ T cells from normal mice were labelled with 2.5 μM CFSE in vitro. The CFSE-labelled cells were resuspended in complete RPMI 1640 culture medium at 1×10⁶/ml, and transferred into a 96-well plate coated with 10 μg/ml anti-CD3E antibody at 4° C. overnight. The B cells purified from draining lymph nodes and the foregoing T cells (1:1) were co-cultured for 72 hours in the foregoing 96-well plate containing 1 μg/ml anti-CD28 mAb for use in flow cytometry assays. The proliferation of the CFSE-labelled T cells was calculated using the equation: (1-MFI value of CFSE of T Cells in Experimental Group/MFI value of CFSE of B-cell-free T Cells in Control Group)×100/a.

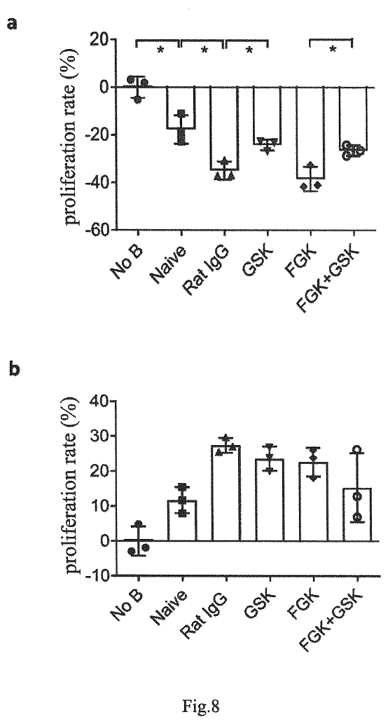

When CD4⁺ T cells were cultured with CD19⁺CD24^{hi}IgD^{lo/-} B cells, proliferation of the activated T cells was suppressed. The tumor factor increased suppressive effects of CD19⁺CD24^{hi}IgD^{lo/-} B cells in draining lymph nodes of mice. FGK had no significant effects on the suppressive effects of CD19⁺CD24^{hi}IgD^{lo/-} B cells in draining lymph nodes of tumor-bearing mice, and GSK reduced suppressive effects of CD19⁺CD24^{hi}IgD^{lo/-} B cells in draining lymph nodes of tumor-bearing mice (FIG. 8a). In the co-culture system of T cells and the CD19⁺CD24^{lo}IgD^{hi} B cell population, CD19⁺CD24^{lo}IgD^{hi} B cell population promoted but not inhibited proliferation of activated T cells (FIG. 8b).

With the results recited above, we found that CD19⁺CD24^{hi}IgD^{lo/-} B cells expressed higher PPARδ, thus having immunosuppressive effects. The PPARδ inhibitor mainly reduced proliferation of CD19⁺CD24^{hi}IgD^{lo/-} B cells caused by tumors and FGK45.5, reduced increase in suppressive effects of CD19⁺CD24^{hi}IgD^{lo/-} B cells caused by tumors, thereby, at animal level, reducing immunosuppressive effects of CD19⁺CD24^{hi}IgD^{lo/-} B cells, promoting immunotherapy effects of an immune stimulating antibody or and immune checkpoint antibody on tumors.

### Example 9: Therapeutic Effects of PPARδ Inhibitor Combined with FGK45.5 on Mouse MB49 Bladder Cancer

From the results above, we anticipate that the PPARδ inhibitor could also enhance therapeutic effects of immunotherapeutic drug on tumors in other tumor models. In order to verify such possibility, we examined the therapeutic effects of the PPARδ inhibitor in combination with FGK45.5 on MB49 bladder cancer cell tumor-bearing mice. C57BL/6 mice (8 to 10 weeks, n=5/group, 4 groups) are subcutaneously inoculated with 7×10⁵ MB49 cells (mouse bladder cancer cells) and about 8 days after inoculation of tumor cells when tumors became touchable, Rat IgG, or FGK45.5, or PPARδ inhibitor (GSK3787), or FGK45.5 in combination with PPARδ inhibitor (GSK3787) was administered. Tumor growth condition was monitored. Doses and regimens of Rat IgG, FGK45.5 and the PPARδ inhibitor (GSK3787) in all groups followed the doses and regimens of Example 2, as shown in FIG. 2a.

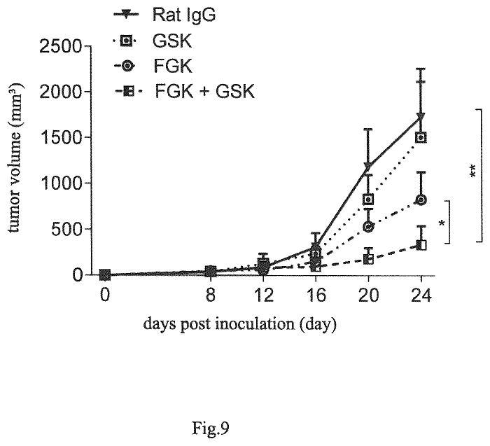

It was found that FGK45.5 (anti-CD40 agonistic antibody of mouse) used solely has moderate therapeutic effects on MB49 tumors, while GSK3787 (PPARδ inhibitor) used solely has no therapeutic effects on MB49 tumors, but the use of GSK3787 could further improve the effect of FGK45.5 in treating MB49 tumors (FIG. 9).

### Example 10: PPARδ Inhibitor could Reduce Immunosuppression of CD19⁺CD24^{hi}IgD^{lo/+} B Cell Subpopulation from Non-Small Cell Lung Cancer (NSCLC) Patients (Adenocarcinoma and Squamous Cell Carcinoma)

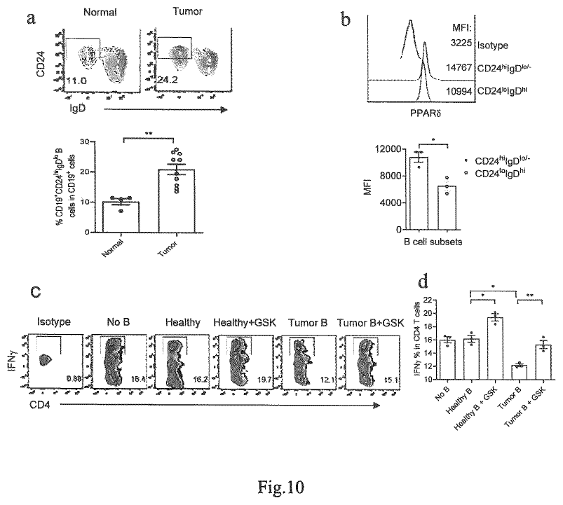

For the purpose of further proving the clinical significance of our researches, peripheral blood of non-small cell lung cancer (NSCLC) patients (including adenocarcinoma and squamous cell carcinoma patients) was sampled, flow cytometry of which revealed that: similar to the findings on mouse models, compared with healthy subjects, proportion of CD19⁺CD24^{hi}IgD^{lo/-} B cells in the peripheral blood of cancer patients significantly increased, and human CD19⁺CD24^{hi}IgD^{lo/-} B cells expressed higher level of PPARδ than human CD19⁺CD24^{lo}IgD^{hi}B cells (FIG. 10b).

B cells were isolated and gathered from the peripheral blood of multiple normal blood donors or patients, and CD19⁺CD24^{hi}IgD^{lo/-} B cells were isolated from the gathered B cells and cultured for 24 hours in complete RPMI 1640 culture medium with or without the PPARδ inhibitor GSK3787. Then these B cells were washed twice to remove the PPARδ inhibitor, resuspended in complete RPMI 1640 culture medium at 1×10⁶/ml. CD4⁺CD25⁻ T cells were isolated from blood of normal blood donors. The treated CD19⁺CD24^{hi}IgD^{lo/-} B cells and these T cells (1:1) were co-cultured for 48 hours, Dynabeads were coated with anti-human CD3 mAb and anti-human CD28 mAb and placed on a 96-well U-shaped culture plate. Golgi^{plug} (BD Biosciences), PMA and ionomycin were added into the culture medium together for 6 hours. CD4⁺ T cells were surface stained, perforated in the cytomembrane, and stained intracellularly using fluorescence labelled anti-human IFNγ, then analyzed using flow cytometry. The analysis revealed that CD19⁺CD24^{hi}IgD^{lo/-} B cells from cancer patients exhibited stronger ability to suppress IFNγ secretion of T cells activated by anti-human CD3 mAb and anti-human CD28 mAb than the CD19⁺CD24^{hi}IgD^{lo/-} B cells from the healthy control group (FIG. 10c). In addition, suppressive activity of human CD19⁺CD24^{hi}IgD^{lo/-} B cells of healthy subjects and cancer patients were significantly reduced by the GSK3787 treatment. These results show that, similar to the case of mice, human CD19⁺CD24^{hi}IgD^{lo/-} B cells are also a critical cancer-related B cell subset having a suppressive function, the immunosuppressive function of which depends on PPARδ to a great deal, and the PPARδ inhibitor could reduce the immunosuppressive function of CD19⁺CD24^{hi}IgD^{lo/-} B cells from cancer patients.

It should be noted that the above-mentioned specific embodiments are exemplary, and those skilled in the art can come up with various solutions inspired by the disclosure of the present invention, and these solutions also belong to the disclosure scope of the present invention and fall within the scope of the present invention. It should be understood by those skilled in the art that the description of the present invention and the accompanying drawings are illustrative rather than limiting to the claims. The protection scope of the present invention is defined by the claims and their equivalents.

1-15. (canceled)

16. A PPARδ inhibitor in combination with an immunotherapeutic drug for use in the treatment of tumor, wherein the tumor is preferably melanoma, mammary cancer, ovarian cancer, pancreatic cancer, lung cancer, liver cancer, esophageal cancer, colorectal cancer, colonic cancer, lymphoma, brain tumor, sarcoma, cervical cancer, prostate cancer, bladder cancer, osteosarcoma, head and neck cancers, renal cell carcinoma, or stomach cancer.

17. The PPARδ inhibitor in combination with an immunotherapeutic drug according to claim 16, wherein the immunotherapeutic drug is an immune agonist or an immune checkpoint inhibitor.

18. The PPARδ inhibitor in combination with an immunotherapeutic drug according to claim 17, wherein the immune agonist is an agonist specific for costimulatory molecules, which include OX40, 4-1BB(CD137), CD27, GITR, CD28, and/or ICOS.

19. The PPARδ inhibitor in combination with an immunotherapeutic drug according to claim 18, wherein the immune agonist is a CD40 agonist.

20. The PPARδ inhibitor in combination with an immunotherapeutic drug according to claim 19, wherein the immune checkpoint inhibitor is selected from a PD-1 inhibitor, a PDL1 inhibitor, a TIM3 inhibitor, a LAG3 inhibitor, a CD47 inhibitor; and preferably the immune checkpoint inhibitor is selected from an anti-PD-1 antibody, an anti-PDL1 antibody, an anti-TIM3 antibody, an anti-LAG3 antibody, an anti-CD47 antibody, and an anti-CTLA-4 antibody.

21. The PPARδ inhibitor in combination with an immunotherapeutic drug according to claim 20, wherein the immune checkpoint inhibitor is an anti-PD-1 antibody.

22. The PPARδ inhibitor in combination with an immunotherapeutic drug according to claim 21, wherein the CD40 agonist is selected from an anti-CD40 agonistic antibody, a CD40L protein, an expression vector of a CD40L protein, or a fragment, a derivative, and/or a polymer thereof; preferably the CD40L protein is a recombinant CD40L protein; and preferably the CD40 agonist is an anti-CD40 agonistic antibody.

23. The PPARδ inhibitor in combination with an immunotherapeutic drug according to claim 22, wherein the PPARδ inhibitor is a compound being able to inhibit PPARδ, or a nucleic acid molecule being able to inhibit effects of mRNA of PPARδ, or a molecule being able to decompose PPARδ in a targeted manner; preferably the nucleic acid molecule is siRNA or shRNA; and
preferably the PPARδ inhibitor is GSK3787.

24. An anti-tumor drug composition, comprising a PPARδ inhibitor and an immunotherapeutic drug, wherein the PPARδ inhibitor and an immunotherapeutic drug are prepared separately and then packaged together, or a formulation prepared by mixing the PPARδ inhibitor and the immunotherapeutic drug together; the tumor is preferably melanoma, mammary cancer, ovarian cancer, pancreatic cancer, lung cancer, liver cancer, esophageal cancer, colorectal cancer, colonic cancer, lymphoma, brain tumor, sarcoma, cervical cancer, prostate cancer, bladder cancer, osteosarcoma, head and neck cancers, renal cell carcinoma, or stomach cancer; the tumor is preferably melanoma, bladder cancer, or non-small cell lung cancer (NSCLC).

25. The anti-tumor drug composition according to claim 24, wherein the PPARδ inhibitor is a compound being able to inhibit PPARδ, or a nucleic acid molecule being able to inhibit effects of mRNA of PPARδ, or a molecule being able to decompose PPARδ in a targeted manner.

26. The anti-tumor drug composition according to claim 25, wherein the nucleic acid molecule is siRNA or shRNA; and preferably the PPARδ inhibitor is GSK3787.

27. The anti-tumor drug composition according to claim 26, wherein the immunotherapeutic drug is an immune agonist or an immune checkpoint inhibitor.

28. The anti-tumor drug composition according to claim 27, wherein the immune agonist is an agonist specific for costimulatory molecules, which include OX40, 4-1BB(CD137), CD27, GITR, CD28, and/or ICOS; preferably the immune agonist is a CD40 agonist.

29. The anti-tumor drug composition according to claim 28, wherein the immune checkpoint inhibitor is selected from a PD-1 inhibitor, a PDL1 inhibitor, a TIM3 inhibitor, a LAG3 inhibitor, a CD47 inhibitor; and preferably the immune checkpoint inhibitor is selected from an anti-PD-1 antibody, an anti-PDL1 antibody, an anti-TIM3 antibody, an anti-LAG3 antibody, an anti-CD47 antibody, and an anti-CTLA-4 antibody.

30. The anti-tumor drug composition according to claim 29, further comprising a pharmaceutically acceptable carrier, and being prepared into a pharmaceutically acceptable formulation;
preferably the formulation is an injection, a targeting formulation, or a nano-formulation.

31. A method for treating tumor, wherein method comprises the steps of administering to a subject in need of such treatment an effective amount of
(1) a PPARδ inhibitor; and
(2) an immune stimulator or immune checkpoint inhibitor,
wherein (1) and (2) may be administered simultaneously or separately.

32. The method according to claim 31, wherein the cancer is melanoma, bladder cancer, or bladder cancer, or non-small cell lung cancer (NSCLC).

33. The method according to claim 32, wherein the PPARδ inhibitor comprises a small molecule inhibitor of PPARδ, preferably the PPARδ inhibitor comprises siRNA or shRNA, preferably the PPARδ inhibitor comprises GSK3787.

34. The method according to claim 33, wherein the PPARδ inhibitor may be administered orally, intramuscularly, intravenously, or intraperitoneally.

35. The method according to claim 34, wherein the immune stimulator or immune checkpoint inhibitor is a CD40 stimulator, preferably the CD40 stimulator is an anti-CD40 antibody.
